(19) United States
(12) Patent Application Publication
(10) Pub. No.: US 2007/0283453 A1
(43) Pub. Date: Dec. 6, 2007

(54) ANIMAL MODELS OF TUMOR METASTASIS AND TOXICITY

(75) Inventors: Juan Carlos Rodriguez Cimadevilla, Tres Cantos Madrid (ES); Isabel Punzon Gau, Tres Cantos Madrid (ES)

Correspondence Address: INTELLECTUAL PROPERTY / TECHNOLOGY LAW, PO BOX 14329, RESEARCH TRIANGLE PARK, NC, 27709, US

(73) Assignee: Juan Carlos Rodriguez Cimadevilla, Tres Cantos Madrid (ES)

(21) Appl. No.: 11/756,233

(22) Filed: May 31, 2007

Related U.S. Application Data
(60) Provisional application No. 60/809,306, filed on May 31, 2006.

(51) Int. Cl. A01K 67/027

(52) U.S. Cl. 800/10

(57) ABSTRACT

A method for conversion of an animal into an appropriate recipient of tumor cells derived from a different species. Animals for such purpose can be immuno-incompetent animals that are doubly grafted with orthotopic tissues, in which one grafted tissue (i.e., breast) is from an organ of the same class as the tumor of origin (graft A), and the second grafted tissue (i.e., bone) is from a organ of the same class as a target organ for metastasis (graft B). These dual grafted animals can be used to model human diseases. In one implementation, human tumor cells are orthotopically seeded in graft A in order to analyze the occurrence of metastasis in graft B. Methods and compositions are described for creating a multiorgan human environment in mice, by grafting human stem cells (mesenchymal, embryonic or others) into mice, e.g., injured to enhance specific tissue engraftment. Such chimeric mice can be used to grow human tumors and to study the occurrence of metastasis.

### CROSS-REFERENCE TO RELATED APPLICATION

This claims the benefit of priority under 35 USC §119 of U.S. Provisional Patent Application No. 60/809,306 filed May 31, 2006. The entire disclosure of said U.S. Provisional Patent Application No. 60/809,306 is hereby incorporated herein by reference in its entirety for all purposes.

### FIELD OF THE INVENTION

The invention relates to the field of animal metastasis models and, in particular, to an animal metastasis model wherein the organ which is the target for metastasis is from a different species than the animal and has been reconstituted by damaging the corresponding organ in the animal prior to the administration of stem cells. Moreover, the invention relates also to the field of animals carrying xenoorgans and the uses thereof in toxicity models.

### BACKGROUND OF THE INVENTION

Metastasis (Greek: change of the state, plural: metastases), sometimes abbreviated Mets, is the spread of cancer from its primary site (source tissue) to other places in the body (the target tissue).

Metastatic tumors are very common in the late stages of cancer. The spread of metastases may occur via the blood or the lymphatics or through both routes. The most common places for the metastases to occur are the adrenals, liver, brain and the bones. There is also a propensity for certain tumors to seed in particular organs. This was first discussed as the “seed and soil” theory by Stephen Paget over a century ago in 1889. For example, prostate cancer usually metastasizes to the bones. Similarly, colon cancer has a tendency to metastasize to the liver. Stomach cancer often metastasizes to the ovary in women, where it forms a Krukenberg tumor.

It is difficult for cancer cells to survive outside their region of origin, so in order to metastasize they must find a location with similar characteristics. For example, breast tumor cells, which gather calcium ions from breast milk, metastasize to bone tissue, where they can gather calcium ions from bone. Malignant melanoma spreads to the brain, presumably because neural tissue and melanocytes arise from the same cell line in the embryo. When cancer cells spread to form a new tumor, it is called a secondary or metastatic tumor, and its cells are like those in the original tumor. This means, for example, that if breast cancer spreads (metastasizes) to the lung, the secondary tumor is made up of abnormal breast cells (not abnormal lung cells). The disease in the lung is metastatic breast cancer (not lung cancer).

Animal models are important tools to investigate the pathogenesis and develop treatment strategies for metastases in humans. Animal models which have been successively used as models for metastasis include spontaneous tumor models, human tumor xenografts (sub-renal capsule or subcutaneous), syngenic tumor cell injections (intravenous, subcutaneous or intracardiac), orthotopic transplantation of human tumors or cell lines thereof, autochthonous animal tumors or genetically engineered cancer models.

The present invention relates to two of these animal models, namely, human tumor xenografts and orthotopic transplantation of human tumors or cell lines.

It is known from US2005132427, U.S. Pat. No. 6,365,797 and US2002199212 that immunodeficient animals, in particular, NOD/SCID mice, when inoculated with a tumor cell from a human metastatic tumor or a human tumor cell line are suitable animal models for metastasis. Alternatively, animal models can be obtained by implantation of human tumor cells, either directly into the metastasis-target tissue (orthotopic implantation) or at a different location, i.e. subcutaneous, as it has been described in the international patent application WO9816628. Moreover, it is known from US2006123494 that human mammary epithelial cells can produce tumors when transduced with HER2/SV40 early region and mixed with stromal fibroblasts and HGF at the time of implantation.

However, the use of these models for studying the metastases of human cancer cells has so far been limited, due to (i) the low efficiency of the incidence of cancer metastasis in the recipient mice, usually due to the fact that the transplanted material consists of tumor derived cell lines that often are cells that have been over transformed in vitro after many uncontrolled passages and (ii) the large cell number required to achieve the desired results. A possible solution to this problem is the use of a specific cell population present within solid epithelial tumors that, when injected into the animal, can recapitulate the whole process of tumor development. These cells (also called cancer stem cells) have been well characterized in human breast cancer but are difficult to isolate.

An alternative type of animal models for metastasis which overcomes the above problems are the double engraftment models wherein the animal receives two grafts: One grafted tissue (i.e. breast) is from a human organ of the same class as the tumor of origin (graft A) and a second grafted tissue (i.e. bone) is from a human organ of the same class as a target organ for metastasis (graft B). These dual grafted mice can be used to model human diseases. A preferred model will be one in which human tumor cells are orthotopically seeded in graft A in order to analyze the occurrence of metastasis in graft B.

This type of animal models is known in the art. For instance, Shtivelman, E and Namikawa, R. (Proc. Natl. Acad. Sci. USA, 1995, 92:4661-4665) and U.S. Pat. No. 5,643,551 describe a model for lung cancer metastasis into lungs and bone marrow wherein immunodeficient mice (SCID) are first grafted with human foetal lung and human fetal bone marrow and secondly injected with human small cell lung cancer cells. Similarly, Urashima, M. et al. (Blood, 1997, 90:754-765) describe a model for the homing of multiple myeloma cells to bone marrow wherein SCID-mice are first implanted with human fetal bone grafts and second injected with multiple myeloma cells. Yonou, H. et al. (Cancer Res. 2001 Mar. 1; 61(5):2177-82) describe a mouse model for metastasis of prostate cancer wherein SCID mice are engrafted with human adult bone (HAB) and lung (HAL) followed by injection of cells derived from a prostate cancer cell line. Nemeth, J. A. et al. (Cancer Res., 1999, 59:1987-93) also describe a mouse model for metastasis of prostate cancer into bone, lung and intestine wherein SCID mice are engrafted with human adult bone, lung intestine and then either injected or implanted orthotopically with human prostate cancer cells.

However, this type of models poses different challenges which make them not easy to implement. In particular, it is required that (i) the mouse tissue hosting the implant should have anatomical, structural and molecular characteristics similar to the equivalent human tissue, (ii) the mouse pharmacological features (biodistribution, metabolism, catabolism, clearance, etc.) should reproduce, as faithfully as possible, human pharmacology and (iii) the mouse target tissues for tumor metastasis should also be reasonably good matches for the human equivalent organs (especially those organs among the most frequent human metastasis targets: bone, liver, lung, or brain).

This problem can be overcome, at least partially, by providing the recipient animal, instead of with the organ or tissue as a xenograft, with precursor or stem cells to said target organ or tissue which, ideally, should migrate to the organ concerned and differentiate to produce the target organ. Although it has been demonstrated that human stem cells injected into the mouse blood stream, can localize to multiple organs and have the capacity to recapitulate the local cellularity of each organ, these methods are faced with the problem that the transplanted stem cells, depending on the injection location or method, localize to a variety of organs, so that it is often difficult reconstitute in the recipient animal the organ of interest.

Therefore, there is a need in the art for methods which would allow directing injected stem cells to a particular location of interest (i.e. breast, prostate, etc.).

### SUMMARY OF THE INVENTION

The present invention is based on the surprising finding that the targeting efficiency of the stem cells into an organ of choice can be substantially improved if the recipient animal, prior to receiving the tumor cells, has been treated so as to inflict damage leading to cellular death to the tissue in the recipient organism which corresponds to the target tissue is damaged. In this manner, the damaged organ can be sensed by the implanted stem cells that migrate to the site of damage, undergo differentiation, and promote structural and functional repair or that organ. The animals carrying said reconstituted organ are exceptionally useful as experimental models to study metastasis processes as well as for studying interactions between exogenous compounds and said organs.

Therefore, in one aspect, the invention relates to a method for obtaining an animal model for tumor metastasis comprising at least one cellular component from at least one other animal from a different species comprising the steps of
i) inflicting damage leading to cellular death to at least one tissue or organ in a recipient animal,
ii) implanting into the recipient animal a composition comprising precursor or stem cells from a donor animal from a different species wherein said precursor or stem cells are capable of differentiating into the same tissue, tissues, organ or organs that have been damaged in the recipient animal in step (i),
iii) allowing the cells implanted in step (ii) to differentiate into the at least one tissue or at least one organs that has or have been damaged in step (i) and
iv) implanting into the recipient animal cells derived from a tumor isolated from the donor animal or cells of a tumor cell line which derive from a tumor isolated from the donor animal.

In a second aspect, the invention relates to a non-human animal obtained according to the method of the invention.

In another aspect, the invention relates to the use of a non-human animal of the invention for the study of metastases.

In a further aspect, the invention relates to a method for the identification of a substance capable of inhibiting and/or preventing metastasis of tumor cells, comprising the steps of:
i) administering a test substance to a non-human animal of the invention and
ii) measuring inhibitory and/or preventive effect of the test substance on metastasis.

In a further aspect, the invention provides a method for evaluating efficiencies of treatment against metastasis of tumor cells, comprising the steps of:
i) applying a treatment to the non-human animal of the invention and
ii) comparing the size and/or extent of metastasis, and/or symptoms resulted from metastasis, with a control animal.

In a further aspect, the invention provides a method for identifying genes which are involved in metastasis progression comprising the steps of
i) Preparing a non-human animal of the invention wherein the tumor cells implanted in step (iv) have been transfected with a cDNA whose effect in metastasis wants to be studied.
ii) Monitoring the appearance of metastatic lesions in the organ which has been regenerated in step (iii)
   wherein the appearance of a higher number of metastatic lesions in the animal in comparison with a control animal which has received non-transformed tumor cells is indicative that the candidate gene is involved in metastasis progression.

In a further aspect, the invention provides a method for determining the effect of a test substance on metastasis, comprising the steps of:
i) administering a test substance to a non-human metastasis model animal according to the invention; and
ii) comparing the size and/or extent of metastasis, and/or symptoms resulted from metastasis, with a control animal.

In yet another aspect, the invention provides the use of an animal comprising at least one cellular component from at least one other animal from a different species obtainable by a process comprising the steps of
i) inflicting damage leading to cellular death of at least one tissue or organ in a recipient animal,
ii) implanting into the recipient animal a composition comprising precursor or stem cells from a donor animal from a different species wherein said precursor or stem cells are capable of differentiating into the same tissue, tissues, organ or organs that have been damaged in the recipient animal in step (i) and
iii) allowing the cells implanted in step (ii) to differentiate into the tissue, tissues, organ or organs that have been damaged in step (i) so as to regenerate at least in part the organ that has been damaged in step (i) to evaluate the interaction or effects of a compound of interest with or on the organ or tissue that has been regenerated in the recipient animal.

### BRIEF DESCRIPTION OF THE DRAWINGS

FIG. 1 illustrates a schematic representation of the methods used in the preparation of the strochimeric animals. The method comprises the steps of #1 Bone marrow extraction, #2 isolating the bone marrow-derived mononuclear cells, #3 immortalization of MSC lines, #4 systemic (i.v., i.p., i.c., etc.) injection of primary or immortalized MSC lines, #5 orthotopic graft of human adult epithelial stem cells, #6 orthotopic graft of human cancer cells.

FIG. 2 illustrates an alternative embodiment for the preparation of the strochimeric animals. The method comprises the steps of #1 bone marrow extraction, #2 seeding the bone marrow mononuclear cells, #3 Immortalization of MSC lines, #4 orthotopic graft of primary or immortalized MSC lines, #5 orthotopic graft of human adult epithelial stem cells, #6 orthotopic graft of human cancer cells.

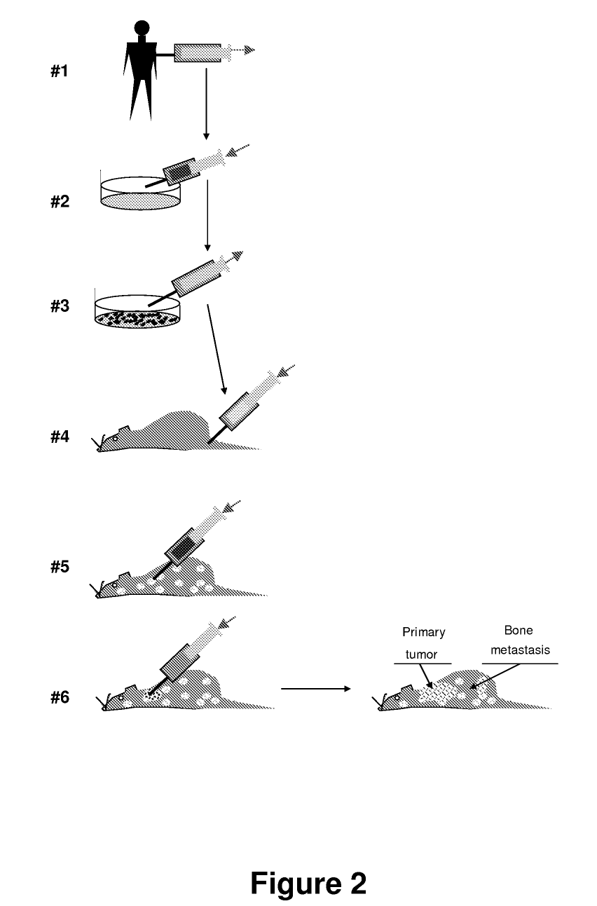

FIG. 3 illustrates the method for preparing the ischimeric animals. The method comprises the steps of #1 preparing immortalized MSC line, #2 performing organ-specific injury (i.e. fat pad, lung, liver, etc.), i.e. ischemia-reperfusion, #3 systemic (i.v. i.p., i.c., etc.) injection of human primary or immortalized MSC lines, #4 systemic injection of orthotopic graft of human adult epithelial stem cells and #5 systemic injection (or orthotopic graft) of human cancer cells.

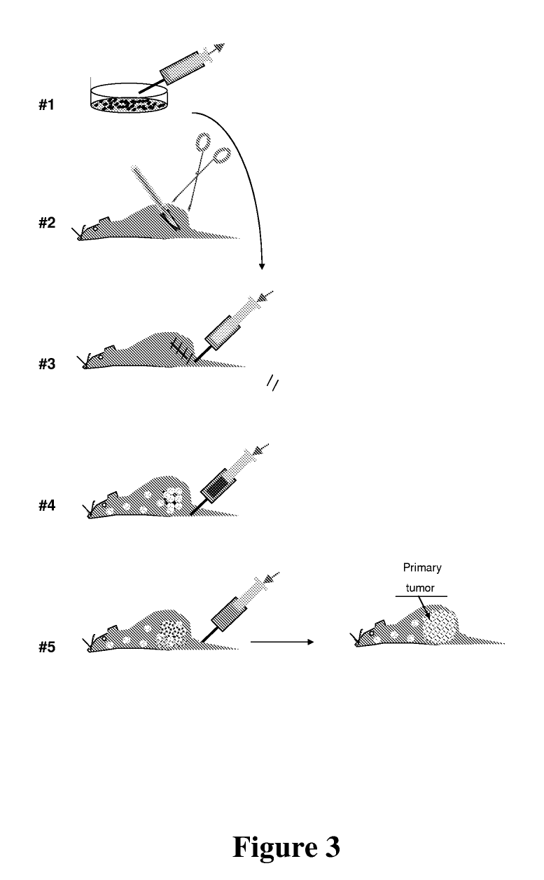

### DETAILED DESCRIPTION OF THE INVENTION

The term “stem cells”, as used herein, defines any precursor cell which is capable of differentiating into one or more of the tissues that are found in the adult animal. Therefore, stem cells is used interchangeably to refer to “stem cells” as such which retain the ability to renew themselves through mitotic cell division and can differentiate into a wide range of specialized cell types, as well as to “precursor cells” (also known as “progenitor cells”), which have only limited capability to proliferate and retain their capability to differentiate but only onto a narrower range of specialized cell types.

“Organ” as used herein, refers to a differentiated part of an organism which with a specific function. Examples include, but are not limited to, parts which have specific functions such as respiration, secretion or digestion.

The term “ischimeric animal”, as used herein, relates to a chimeric animal in which the xenoorgan has been produced by previously inflicting ischemic damage to the corresponding organ in the recipient organism, so as to promote repopulation of stem cells.

The term “mesenchimeric animal” as used herein, relates to chimeric animals which are, prior or simultaneously to the administration of the stem cells, provided with mesenchimeric stein cells so as to obtain a matrix within the damaged or an which serves as a substrate for the stem cells to differentiate into the cells of the damaged organ.

The term “strochimeric animal”, as used herein, relates to chimeric animals which contain a xenorgan resulting from the repopulation of a damager organ by stem cells of a different species.

The term “fibrochimeric animal”, as used herein, relates to chimeric animals which are, prior or simultaneously to the administration of the stem cells, provided with fibroblasts so as to generate a matrix within the damaged organ which serves as a substrate for the stem cells to differentiate into the cells of the damaged organ.

In a first aspect, the invention provides a method for obtaining an animal model for tumor metastasis comprising the steps of
(i) inflicting damage leading to cellular death to at least one tissue or organ in a recipient animal,
(ii) implanting into the recipient animal a composition comprising precursor cells from another organism from a different species which are capable of differentiating into the same at least one tissue or organ that has been damaged in the recipient animal in step (i),
(iii) allowing the cells implanted in step (ii) to differentiate into the at least one tissue or organ that has been damaged in step (i) and
(iv) implanting into the recipient animal cells derived from a tumor isolated from the donor animal or cells of a tumor cell line which derive from a tumor isolated from the donor animal.

Suitable animals that can be used as recipient animals for the present invention include any species, preferably mammals and, more preferably primate (monkey, baboon, chimpanzee and the like), rodent (mouse, rat, rabbit, guinea pig, hamster, and the like) or a pig. In a preferred embodiment, the recipient animal is a rodent, more preferably a mouse.

In a preferred embodiment, the recipient animal is an immunodepressed animal. Severe combined immune deficient (SCID) mice are the preferred recipient animals utilized in the practice of the invention. Various other immune deficient mice, rodents or animals may be used, including those which are deficient as a result of a genetic defect, which may be naturally occurring or induced, such as, for example, nude mice, Rag 1 and/or Rag 2 mice, and the like, and mice which have been cross-bred with these mice and have an immunocompromised background. The deficiency may be, for example, as a result of a genetic defect in recombination, a genetically defective thymus or a defective T-cell receptor region. Induced immune deficiency may be as a result of administration of an immunosuppressant, e.g. cyclosporine, removal of the thymus, etc. Various transgenic immune deficient mice are currently available or can be developed in accordance with conventional techniques. Ideally, the immune deficient mouse will have a defect which inhibits maturation of lymphocytes, particularly lacking the ability to rearrange the T-cell receptor region. In the particular and preferred embodiments described herein, the immunodepressed animal is a SCID mouse or a NOD-SCID mouse. In addition to mice, immune deficient rats or similar rodents may also be employed in the practice of the invention.

The animals that can be used as recipient animals can be in any developmental stage. In a preferred embodiment, the recipient animal is in the embryonic stage.

As donor animal from which the tumor and the stem cells are isolated and implanted into the recipient animal, any animal which could benefit from the present method can be used for the purposes of the present invention provided that it is from a different species than the recipient animal. This includes, without limitation, humans and non-human primates, livestock animals, companion animals, laboratory test animals, captive wild animals, reptiles and amphibians, fish, birds and any other organism.

In another embodiment, the tissues and organs in the recipient animal are damaged by means of chemical agents, more particularly, by means of a cytotoxic agent. Cytotoxic agents that can be used in the present invention, include, without limitation, radionuclides, either administered on its own or coupled to an antibody to achieve specific delivery to a particular tissue. Suitable radionuclides are e.g., actinium (²²⁵Ac), astatine (²¹¹At), bismuth (²¹³Bi or ²¹²Bi), carbon (¹⁴C), cobalt (⁵⁷Co), copper (⁶⁷Cu), fluorine (¹⁸F), gallium (⁶⁸Ga or ⁶⁷Ga), holmium (⁶⁶Ho), indium (¹¹⁵In, ¹¹³In, ¹¹²In, or ¹¹¹In), iodine (¹³¹I, ¹²⁵I, ¹²³I, or ¹²¹I), lead (²¹²Pb), lutetium (¹⁷⁷Lu), palladium (¹⁰³Pd), phosphorous (³²P), platinum (^{195m}Pt), rhenium (⁸⁶Re or ¹⁸⁸Re), rhodium (¹⁰⁵Rh), ruthenium (⁹⁷Ru), samarium (¹⁵³Sm), scandium (⁴⁷Sc), technetium (^{99m}Tc), ytterbium (¹⁶⁹Yb or ¹⁷⁵Yb), or yttrium (⁹⁰Y). In other embodiments, the cytotoxic agent is a chemotherapeutic agent or toxin (e.g., cytostatic or cytocidal agent). Examples of chemotherapeutic agents and toxins include the following non-mutually exclusive classes: alkylating agents, anthracyclines, antibiotics, antifolates, antimetabolites, antitubulin agents, chemotherapy sensitizers, DNA minor groove binders, DNA replication inhibitors, duocarmycins, etoposides, fluorinated pyrimidines, lexitropsins, microbial and plant toxins, nitrosoureas, platinols, purine antimetabolites, puromycins, steroids, taxanes, topoisomerase inhibitors, and vinca alkaloids. In another embodiment, the cytotoxic agent is a toxins such as enzymatically active toxins of bacterial, fungal, plant or animal origin, or fragments thereof. Suitable toxins and their corresponding fragments include diphtheria A chain, exotoxin A chain, ricin A chain, abrin A chain, curcin, crotin phenomycin, enomycin, dolastatin 10, auristatins, such as auristatin E and auristatin F, calicheamicin, and the like.

In another embodiment, the invention provides methods wherein the damage in step (i) is caused by physical means. Preferably, the damage is caused by ischemia-reperfusion without producing organ failure. Cell death and tissue damage caused by ischemia-reperfusion can be reasonably predicted to occur as a result of certain surgical procedures like, for instance, balloon angioplasty, coronary bypass surgery, heart transplantation, and valve replacement surgery. Similar damage occurs in the kidney, liver, and other organs resulting from decrease or cessation of blood flow. A preferred type of ischemia is that caused after clamping an afferent vessel. Arteries irrigating an organ that are candidates for clamping can be of the type of main organ arteries or subsidiary arteries. The choice of one or the other type will depend on whether one can afford functional organ insufficiency (usually in the case of double organs like kidney, lung, etc.) or not. In double or multiple organs like kidney, lungs, breast, ovaries, etc., ischemia by clamping the main afferent artery can by a valid non-exclusive option. The choice of one or the other type will depend on whether one can afford functional organ insufficiency (usually in the case of double organs like kidney, lung, etc.) or not (see below).

In another embodiment, the present invention provides a method for obtaining a animal model of metastasis wherein only one organ of the recipient animal is damaged and, consequently, only one organ of the donor animal will be regenerated in the recipient animal. In principle, any organ or tissue that may be the target for metastasis can be used in the model according to the present invention, provided that said organ or tissue can be specifically damaged or injured by means and provided that said organ or tissue can be reconstituted in the recipient animal by implanting the corresponding precursor cells. Organs which can be preferably employed in the present invention as metastasis models include organs of any region of the body, including head and neck (face, orbit, eye, mouth, tongue, teeth, nose, ears, scalp, larynx, pharynx, salivary glands, meninges, brain, thyroid and parathyroid gland), back and spine (vertebra and spinal cord), thorax (mammary gland, ribs, lungs, heart, mediastinum, esophagus and diaphragm), abdomen (peritoneum, stomach, duodenum, intestine, colon, liver, kidney, adrenal gland, appendix and pancreas), pelvis (sacrum, coccyx, ovaries, fallopian tube, uterus, vagina, vulva, clitoris, perineum, urinary bladder, testicles, rectum and penis) and limbs (muscle, bone, nerves, hand, wrist, elbow, shoulder, hip, knee or ankle).

In a preferred embodiment, only part of the organ is damaged. For instance, in a preferred embodiment, only one lobe of the liver is damaged using a partial hepatic ischemia model which spares the right lobe, which has the advantage that intestinal congestion, sepsis and peritonitis are avoided.

In yet another embodiment, several of the organs mentioned previously may be damaged simultaneously, either completely or partially. Preferred combinations of organs include liver and bone, liver and lung, liver and kidney, lung and kidney and the like.

In yet another embodiment, the whole animal is pre-treated as a target organ (as an example, whenever animals are treated systemically with an agent that is toxic to cells in general, like a cytotoxic, etc., that cases damage to all or most tissues in the animal).

In another embodiment, the cells implanted in step (ii) are systemically administered. This type of administration is preferred when several organs have been damaged so that a single application suffices to allow delivery of the precursor cells to every organ instead of single orthotopic administrations to every metastasis target organ. Systemic administration can be achieved by tail vein injection, intravenous injection, intraperitoneal injection or intracardiac injection. In another embodiment, precursor cells in step (ii) are orthotopically administered into the damaged tissue by direct intra-organ injection. This later type of administration is preferred in those models wherein only one metastasis-target tissue has been damaged.

In another embodiment, the composition which is implanted into the recipient animal in step (ii) is any composition which contains purified precursor cells. Preferably, precursor cells are hematopoietic stem cells (HSC) which can be obtained from blood, bone marrow or umbilical cord. Accordingly, in one embodiment, the composition containing precursor cells is blood. When desired, the donor animal may be treated prior to the obtention of the blood with one of the methods known in the art to promote mobilization of stem cells from the bone marrow into peripheral blood, like, e.g. treatment with GM-CSF. Precursor cell populations can be recovered by any of the many extraction methods known in the ant and can be used, so long as they can be used to obtain the preparations enriched in progenitor cells from the bulk of the ancillary tissue, components including one or more of the following red cells, platelets, granulocytes and unwanted fluids. Suitable cell extraction methods include one or more of the following known methods: plasmapheresis, centrifugation at defined time and g-force or density gradient centrifugation, centrifugation following the addition of some fluids such as physiological solutions or certain soluble polymers, cellular adherence to plastic, and adherence to reagents used to coat growth surfaces including reagents such as fibronectin, and collagen. In addition, mechanical cell sorting methods can be used and enzymatic methods can be used, as are known.

In another embodiment, the compositions containing HSC is a bone marrow aspirate. The harvested bone marrow is processed to remove blood and bone fragments. Harvested bone marrow can be combined with a preservative and frozen to keep the stem cells alive until they are used.

In another embodiment, the composition comprising purified HSC is a preparation from the umbilical cord.

In yet another embodiment, the precursor cell preparation contains adult stem cells. Said cells are found and maintained in adult tissues by signals found in the local environment—the stem cell niche. When necessary, it can expand to generate a transiently amplified pool of progenitors to re-populate tissues. Stem cell quiescence in the niche, for example, is thought to be regulated by cell adhesion. This is mediated in part by homotypic interaction of cadherins from the surrounding niche and the stem cells, as well as interactions between integrins on stem cells and the extracellular matrix. Adult stem cells that are suitable for generating animals carrying the target tissue are, in particular, epithelial stem cells, mesenchymal stem cells and cord blood derived stem cells. The adult stem cells to be used in accordance with the present invention can be isolated from any adult tissue, like for instance, skin, liver, lung, breast and the like.

In another embodiment, the precursor cell preparation contains epithelial stem cells. Similar to what occurs in the haematopoietic system, epithelial tissues are subjected to continuous remodelling and renewal in a tightly regulated manner. In recent years, it has become clear that this tissue renewal involves a hierarchy of cells including slowly proliferating stem cells, rapidly proliferating transmit-amplifying cells and various terminally differentiated cells. These putative tissue-specific stem cells have several properties that make them appealing as targets for transforming genetic events. In particular, they are characterized by a capacity for unlimited self-renewal, and they retain the ability to generate a diverse set of differentiated progeny. In addition, recent work suggests that many signalling pathways thought to be involved in the maintenance of normal stem cells are found to be mutated in human cancers, including those regulated by WNT, beta-catenin, PTEN, TGFbeta, hedgehog, Notch and Bmi-1.

In another embodiment, the composition comprising precursor cells contains mesenchymal stem cells. These are cells which co-purify with the adherent population of mononuclear cells from bone marrow aspirates, have multi-cellular and multi-organ potential under the right environmental conditions (Jiang, Y. et al. 2002, Nature, 418:41-49) and can be isolated not only from bone marrow but also from a variety of mammalian tissues (Rodríguez, A. M., 2005, J. Exp. Med. 1397-1405). Phenotypically defined murine MSC could acquire tissue-specific morphology and antigen expression and thus contribute to different tissue cell-types in vivo (Anjos-Afonso, F. et al., 2004, J. Cell. Science, 117:5565-5664). For instance, purified human MSCs from adult bone marrow engraft in the lungs when injected i.v. (Pelz, O. 2005, Stem Cells, 6:828-833). Purified human MSCs from adult bone marrow engraft in the myocardium and appear to differentiate into cardiomyocytes (Toma, C. et al., 2002, Circulation, 105:93-98). Moreover, intravenous injection of irradiated scid mice with human bone marrow, cord blood, or G-CSF cytokine-mobilized peripheral blood mononuclear cells, results in the engraftment of a human haematopoietic system in the murine recipient (Sitnicka, E., 2003, Blood, 102:881-886).

In another embodiment, the composition comprising purified precursor cells contains embryonic stem cells. These are stem cells derived from the inner cell mass of an early stage embryo known as a blastocyst. Human embryos reach the blastocyst stage 4-5 days post fertilization, at which time they consist of 50-150 cells. ES cells are pluripotent. This means they are able to differentiate into all derivatives of the three primary germ layers: ectoderm, endoderm, and mesoderm. These include each of the more than 220 cell types in the adult body. Pluripotency distinguishes ES cells from multipotent progenitor cells found in the adult since these are only form a limited number of cell types. When given no stimuli for differentiation, (i.e. when grown in vitro), ES cells maintain pluripotency through multiple cell divisions. ES cells are characterized by the presence of different markers which are related to self-renewal and pluripotentiality like, for instance, the transcription factors, OCT4, SOX2 and NANOG, (S. H. Orkin, 2005 Cell 122:828-830) as well as other markers such as but are not limited to the stage-specific embryonic antigen SSEA-3 and SSEA-4 (I. Klimanskaya Y. et al., 2005, Lancet, 365:1636-1641) and the weak expression of tumor rejection antigens such as but are not limited to TRA-1-60 and TRA-1-81.

Embryonic stem cells for use in accordance with the present invention may comprise cells which are obtained from the embryonic tissue formed after gestation (e.g., blastocyst) before implantation (i.e., a pre-implantation blastocyst), extended blastocyst cells (EBCs) which are obtained front a post-implantation/pre-gastrulation stage blastocyst (see WO2006/040763) and embryonic germ (EG) cells which are obtained from the genital tissue of a foetus any time luring gestation, preferably before 10 weeks of gestation.

Methods for obtaining such stent bells and providing initial culture conditions, stack as a liquid culture or semi-solid culture medium, are known in the art. The cells are initially expanded in vivo or in vitro, by contacting the source of the stern cells with a suitable reagent that expands or enriches stem cells in the tissue source or in culture. For example, in the case of hematopoietic stern cells, the donor individual can be treated with an agent that enriches for hematopoietic stern cells and encourages such cells to proliferate without differentiation, such as 5-fluorouracil. Other suitable agents for expansion of a desired stem cell type will be known to those of skill in the art.

In another embodiment, the composition comprising purified progenitor cells contains a mixture of more than one type of precursor cells selected from the group of adult stem cells, embryonic stem cells, umbilical cord stem cells and mesenchymal stem cells.

In yet another embodiment, the stem cells to be implanted into the recipient organism in step (ii) have been immortalized, i.e. the cells have been converted into cells that are capable of indefinite growth without differentiation in a cytokine dependent fashion, while maintaining their ability and potential to differentiate into a number of different lineages under the appropriate conditions. Techniques for inducing cell immortalization include exposure to UV light or to chemical carcinogens, transfection of the cells with oncogenes which include, but are not limited to MYC, ICN-I, hTERT (reverse transcriptase component of the human telomerase), NMYC, S-MYC, L-MYC, Akt (myristoylated). Simian virus 40 (SV40) has been used for some time to immortalize human cells from different tissues in order to gain continuously growing cell lines (Sack, G. H. In vitro, 1981, 17: 1-19). Bone marrow derived MSCs that have been modified with immortalizing genes retain their differentiation potential, or multipotency.

In yet another embodiment, the stem cells used in step (ii) have been genetically modified so as to express a reporter gene that allows tracing the stem cells or any cell originating from said stem cells. Preferred markers according to the present invention are colorigenic markers (e.g. enzymes which catalyse a colour creating reaction), fluorogenic markers, biophotonic markers (e.g. light-producing enzymes, luciferases, reviewed in Doyle et al., Cellular Microbiology 6: 303-317, 2004, or Contag et al., Annu Rev Biomed Eng 4: 235-260, 2002), positron emission tomography markers (e.g., marker enzymes such as herpex simplex virus 1 thymidine kinase which metabolize and, thus, trap, molecules labelled with positron-emitting radioisotopes; e.g., Gambir S S et al., a PNAS 96: 2333-2338, 1999), nuclear magnetic resonance imaging, markers (e.g. protein markers increasing the metal ion content of cells such as ferritin, (Genove G et al., Nature Medicine 11, 450-454, 2005). Preferably, the reporter gene should encode for a protein which is easily detectable upon expression and not be harmful in the animal to be studied. More preferably, the marker should be detectable in living animals upon exposure of said animal to an external signal. Most suitable markers can be selected from Escherichia coli lacZ, any member of the fluorescent protein family including green, cyan, blue, yellow, orange and red fluorescent proteins or human placental alkaline phosphatase (hPLAP, or other marker which allow immediate detection, especially optical detection (by colours, fluorescence. etc.). The markers should not interfere with the metastasis which is to be observed in the model. Therefore, markers which are or are related to transplantation antigens, autoimmune antigens components of the immune system, therapeutically active proteins or peptides or metabolically active proteins are not preferred according to the present invention and completely unsuitable if they confer negative influence on the traceability of the cells in the individual model system (i.e. if the marker interferes with the action of the cells in a disturbing or otherwise negative manner).

In order to achieve an efficient regeneration of the injured organ with the stem cells applied in step (ii), it is necessary to increase the efficiency of the repopulation step. This can be achieved either by increasing the tropism of the stem cells for the injured organ or by facilitating the implantation of the stem cells which have already arrived to the injured organ.

Accordingly, in yet another embodiment of the invention, the recipient animal is treated between steps (i) and (ii) so as to promote the migration of stem cells from distant organs to the place of lesion. In a preferred embodiment, the treatment to promote the migration of stem cells consists on the injection into the target organ of molecules which act as attraction cues of the stem cells to the injured tissues. For instance, integrin □4β1 (VLA-4) promotes the homing of circulating CD34+ bone marrow-derived progenitor cells to the □4β1 ligands VCAM and cellular fibronectin, which are expressed on actively remodelling neovasculature (Jin, H. et al. 2006, J. Clin. Invest. 116:652-662).

In another embodiment, the treatment applied to the recipient animal to increase repopulation of the damaged organ consists on the injection into the injured organ of cell extracts from injured tissues.

In yet another embodiment, the invention provides a method wherein the recipient animal is orthotopically implanted after step (i) with fibroblasts from the donor animal, mesenchymal stem cells or a mixture thereof. The fibroblasts migrate to the interstitial space in the injured organ, wherein they produce matrix proteins that maintain the extracellular scaffold, thus acting as feeder for the later seeding of stem cells into the injured organ. Since it is thought that interstitial fibroblasts derive from MSCs, in another embodiment of the invention, the recipient animals can also be implanted after step (i), systemically or locally (orthotopic) with MSCs, which serve as a feeder for human epithelial stem cells, for cancer cells or for other types of cells that need this kind of cell-cell interaction. MSC are known to migrate to remote tissues and clearly develop a fibroblastic phenotype in culture and thus, their implantation after step (i) results in an increased repopulation of the injured organ by the stem cells.

In another embodiment, the fibroblasts and the MSC are administered as a mixture.

The fibroblasts, mesenchymal cells or the mixture thereof can be administered either orthotopically at the site of tissue damage or systemically. In yet another embodiment, the fibroblasts, mesenchymal cells or the mixture thereof can be administered either simultaneously with the stem cells or prior to the administration of the precursor cells.

In another embodiment, the method according to the invention involves the implantation at the site of organ damage with extracellular matrix (ECM), which is the non-cellular part of a tissue consisting of protein and carbohydrate structures secreted by the resident cells and which serves as a structural element in tissues. In a preferred embodiment, the ECM is a natural matrix which occurs in the damaged tissue.

In still another preferred embodiment, the ECM can be isolated and treated in a variety of ways. When harvested from the tissue source and fabricated into a graft material, the ECMs may be referred to as naturally occurring polymeric scaffolds, bioscaffolds, biomatrices, ECM scaffolds, extracellular matrix material (ECMM), or naturally occurring biopolymers. The ECM materials, though harvested from several different body systems as described below, all share similarities when processed into a graft material. Specifically, since they are subjected to minimal processing after they are removed from the source animal, they retain a stricture and composition nearly identical to their native state. The host cells are removed and the scaffolds may be implanted acellularly to replace or repair damaged tissues white delivering therapeutic agents to the tissue.

In another embodiment, the ECM for use in the present invention can be selected from a variety of commercially available matrices including collagen matrices, or can be prepared from a wide variety of natural sources of collagen. Examples of these naturally occurring ECMs include tela submucosa, acellular dermis, cadaveric fascia, the bladder acellular matrix graft, and amniotic membrane (for review see Hodde J., Tissue Engineering 8(2):295-308 (2002)). In addition, collagen-based extracellular matrices derived from renal capsules of warm blooded vertebrates may be selected for use in preparing the ECM for use in the invention. The ECM derived from renal capsules of warm blooded vertebrates was described iii WO 03/02165. Another type of ECM, isolated from liver basement membrane, is described in U.S. Pat. No. 6,379,710. ECM may also be isolated from pericardium, as described in U.S. Pat. No. 4,502,159. In addition to xenogenic biomaterials, autologous tissue can be harvested as well. Additionally elastin or elastin-like polypeptides (ELPs) and the like offer potential as a biologically active ECM, Another alternative of ECM for use in accordance with the present, invention comprises the collagenous matrix having highly conserved collagens, glycoproteins, proteoglycans, and glycosaminoglycans, and/or growth factors, including Transforming Growth Factor-α, Transforming Growth Factor-β, and/or Fibroblast Growth Factor 2 (basic), in their natural configuration and natural concentration. In another example, the collagenous matrix comprises submucosa-derived tissue of a warm blooded vertebrate, such as small intestine submucosa (SIS) The ECM may be, for example, tela submucosa, “tela submucosa” or “submucosa” refers to a layer of collagen-containing connective tissue occurring under the mucosa in most parts of the alimentary, respiratory, urinary and genital tracts of animals. Tela submucosa is a preferred source of ECM.

In yet another embodiment, the ECM is an artificial cellular matrix such as the commercially available human extracellular matrix (Becton Dickinson) and MATRIGEL® which comprise different combinations of ECM components in natural or processed form.

Moreover, the efficiency of the repopulation of the injured tissue can be improved if the mobilization of the endogenous stem cells is inhibited. Accordingly, in another embodiment, the method of the invention includes an intermediate step between steps (i) and (ii) wherein a treatment which inhibits mobilization of endogenous stem cells is applied to the recipient animal.

In one embodiment, said treatment is done by irradiation. In another embodiment, the treatment is done using nicotine receptor antagonists (U.S. Pat. No. 6,720,340).

In yet another embodiment, the material which is implanted in step (iii) into the recipient organism is a composition comprising at least a tumor cell. Preferably, said material is selected from the group of circulating tumor cells, tumor stem cells, cell lines derived from the immortalization of circulating tumor cells, micrometastatic tumor cells, cell lines derived from the immortalization of micrometastatic tumor cells, cell lines derived from immortalized tumor cells that had been previously purified from solid tumors, primary tumor cells from solid tumors, a piece of fresh tumor that has been resected from a solid tumor, primary tumor cells, cell lines derived from immortalized cells that had been previously purified from clinical metastasis (i.e. the PC3 cell line) and any combination of any of those. In a more preferred embodiment, the cells which are implanted into the recipient animal in step (iii) are tumor stem cells such as those described in WO0212447.

In a preferred embodiment, the cells implanted in step (iii) are genetically modified with a reporter gene so as to express a protein that allows tracing the cells implanted in step (iii). Any type of reporter genes mentioned previously as suitable for tracing the stem cells can be used for this purposes. Preferably, the reporter genes used for the stem cells and for the tumor cells are different, so as to allow simultaneous detection of both cell types in the same recipient animal. In a preferred embodiment, the stem cells applied in step (ii) and the tumor cells applied in step (iii) carry a gene coding for a fluorescent protein which emit light in different regions of the spectrum.

In yet another embodiment, the cancer cells are implanted in step (iii) by a method selected from the group of tail vein injection, intracardial injection, intraperitoneal injection and orthotopic implantation into an organ.

In another aspect, the invention provides a method for obtaining an animal model for tumor metastasis comprising at least one cellular component from at least one other animal from a different species comprising the steps of
(i) implanting into a recipient animal a composition comprising mesenchymal stem cells from a donor animal from a different species and allowing the implanted cells to differentiate into cells of mesenchymal tissues.
(ii) implanting into the recipient animal a composition comprising precursor or stem cells from a donor animal from the same species than the donor animal used in step (i), so that the precursor or stem cells are capable differentiate into the same tissue, tissues, organ or organs that have been populated by the mesenchymal stem cells implanted in step (i) and
(iii) implanting orthotopically into at least one tissue or organ that has been populated by mesenchymal stem cells implanted in step (i) cells derived from a tumor isolated from the donor animal or cells of a tumor cell line which derive from a tumor isolated from the donor animal.

In a preferred embodiment, the mesenchymal stem cells used in the method of the invention are primary cells. In another preferred embodiment, the mesenchymal stem cells are immortalized cells.

In a preferred embodiment, the mesenchymal stem cells are administered systemically. In yet another preferred embodiment, the mesenchymal stem cells are administered orthotopically into a tissue or organ of choice. In a preferred embodiment, the mesenchymal stem cells implanted in step (i) are administered simultaneously with fibroblasts.

Preferably, the material implanted in step (iii) is selected from the group of (i) circulating tumor cells, (ii) cell lines derived from the immortalization of circulating tumor cells, (iii) micrometastatic tumor cells, (iv) lines derived from the immortalization of micrometastatic tumor cells, (v) cell lines derived from immortalized tumor cells that had been previously purified from solid tumors, (vi) primary tumor cells from solid tumors, (vii) a piece of fresh tumor that has been resected from a solid tumor, (viii) primary tumor cells, (ix) cell lines derived from immortalized cells that had been previously purified from clinical metastasis (i.e. the PC3 cell line) and (x) any combination of (i) to (ix).

In another aspect, the invention provides a non-human animal obtainable by any of the methods according to the invention. In one embodiment, the non-human animal obtainable by the methods of the invention has been damaged in step (i) in the liver, kidney, brain, lung and/or bone. In a preferred embodiment, the non-human animal obtainable by the method of the invention is a rodent, preferably, a mouse. In another preferred embodiment, the non-human animal is an immunodepressed animal. In a still more referred embodiment, the non-human animal is a SCID mouse. In another embodiment, the donor animal is a human.

In another aspect, the animals of the invention are used to study metastasis.

In another aspect, the invention provides a method for the identification of a substance capable of inhibiting and/or preventing metastasis of tumor cells, comprising the steps of:
a. administering a test substance to a non-human animal of the invention and
b. measuring inhibitory and/or preventive effect of the test substance on metastasis.

In another aspect, the invention provides a method for evaluating efficiencies of treatment against metastasis of tumor cells, comprising the steps of:
a. applying a treatment to the non-human animal of the invention and
b. comparing the size and/or extent of metastasis, and/or symptoms resulted from metastasis, with a control animal.

In another aspect, the invention provides a method for determining the effect of a test substance on metastasis, comprising the steps of:
a. administering a test substance to a non-human metastasis model animal of the invention; and
b. comparing the size and/or extent of metastasis, and/or symptoms resulted from metastasis, with a control animal.

In these methods, the animals that have developed metastatic cancer can be treated with the test compound(s), and any change in the number, size or other properties of the metastatic nodules as a result of drug treatment, and the viability of the test animals are monitored relative to untreated and/or positive control, where the positive control typically is an animal treated with a know anti-metastatic compound. The administration of the test compounds can be performed by any suitable route, including, for example, oral, transdermal, intravenous, infusion, intramuscular, etc. administration. Results obtained in this model can then be validated by follow-up pharmacokinetic, toxicological, biochemical and immunologic studies, and ultimately human clinical studies.

In another embodiment, the invention provides a method for identifying genes which are involved in metastasis progression comprising the steps of:
a) Preparing a non-human animal according to the invention wherein the tumor cells implanted in step (iv) have been transformed with a cDNA whose effect in metastasis wants to be studied and
b) Monitoring the appearance of metastatic lesions in the organ which has been regenerated in step (iii)
   wherein the appearance of a higher number of metastatic lesions in the animal in comparison with a control animal which has received non-transformed tumor cells is indicative that the candidate gene is involved in metastasis progression.

Moreover, the inventors have made the surprising observation that the animals of the invention containing a xenoorgan resulting from the repopulation of a damaged organ by stem or precursor cells, provide a very accurate model to study the function of said organs closely resembling the physiology of he full organ in the donor anima, being thus very useful for providing insight onto the effect of a given compound on the function of said organ, so that assays carried out in the recipient animal can be dispensed with.

Therefore, in another aspect, the invention relates to the use of an animal comprising at least one cellular component from at least one other animal from a different species obtainable by a process comprising the steps of
(i) inflicting damage leading to cellular death of at least one tissue or organ in a recipient animal,
(ii) implanting into the recipient animal a composition comprising precursor or stem cells from a donor animal from a different species wherein said precursor or stem cells are capable of differentiating into the same tissue, tissues, organ or organs that have been damaged in the recipient animal in step (i) and
(iii) allowing the cells implanted in step (ii) to differentiate into the tissue, tissues, organ or organs that have been damaged in step (i) so as to regenerate at least in part the organ that has been damaged in step (i) to evaluate the interaction or effects of a compound of interest with or on the organ or tissue that has been regenerated in the recipient animal.

In a preferred embodiment, the effect of the candidate compound that is to be evaluated on the regenerated organ is toxicity. Evaluation of toxicity usually involves administering the candidate compound to an animal according to the invention, determining any change in the function of the regenerated organ that is attributable to the compound (compared with untreated animals or animals treated with an inert compound), and then correlating the effect of the compound with the observed change. The method of the invention allows to detect the toxic effects of compound which have a direct effect pharmacological effect on a given organ as well as those compounds which have effects elsewhere that may have unintended hepatic side effects. Moreover, the use of the invention allows to test two or more drugs in combination (by combining with the cells either simultaneously or sequentially), to detect possible drug-drug interaction effects.

In a preferred embodiment, the organ wherein the toxicity is evaluated is liver. Hepatocytotoxicity can be determined in the first instance by the effect of the compound on cell viability, survival, morphology, and leakage of enzymes into the blood stream. More detailed analysis is conducted to determine whether compounds affect cell function (such as gluconeogenesis, ureagenesis, and plasma protein synthesis) without causing toxicity. Leakage of enzymes such as mitochondrial glutamate oxaloacetate transaminase and glutamate pyruvate transaminase can also be used. Other methods to evaluate hepatotoxicity include determination of the synthesis and secretion of albumin, cholesterol, and lipoproteins; transport of conjugated bile acids and bilirubin; ureagenesis; cytochrome P450 levels and activities; glutathione levels; release of a-glutathione S-transferase; ATP, ADP, and AMP metabolism; intracellular K+ and Ca2+ concentrations; the release of nuclear matrix proteins or oligonucleosomes; and induction of apoptosis (indicated by cell rounding, condensation of chromatin, and nuclear fragmentation). DNA synthesis can be measured as [³H]-thymidine or BrdU incorporation. Effects of a drug on DNA synthesis or structure can be determined by measuring DNA synthesis or repair. [³H]-thymidine or BrdU incorporation, especially at unscheduled times in the cell cycle, or above the level required for cell replication, is consistent with a drag effect. Unwanted effects can also include unusual rates of sister chromatid exchange, determined by metaphase spread (see pp. 375-410 of Vickers (1997) In vitro Methods in Pharmaceutical Research Academic Press).

In another embodiment, the organ wherein toxicity is evaluated is lung. Toxicity in the lung can be measured by performing lung function tests, including spirometry, and by measuring pulmonary transfer factor for carbon monoxide (TLCO), the diffusing capacity of the alveolo-capillary membrane (Dm), the pulmonary capillary blood volume (Vc), the transfer factor of the lungs for carbon monoxide per unit alveolar volume (KCO) as well as some biochemical markers such as serum angiotensin-converting enzyme (sACE), serum copper (sCU++) and serum procollagen III peptide (sPIIIP), lactate dehydrogenase, acid phosphatase, alkaline phosphatase and gamma-glutamyl transferase.

In another embodiment, the organ wherein the toxicity is evaluated is kidney. Nephrotoxicity can be evaluated by the use of non-intrussive assays such as serum creatinine and blood urea nitrogen (BUN) levels; creatine clearance rates, urine creatine and protein levels; radioisotope metabolic labelling or soft tissue imaging, including, sonography, magnetic resonance imaging and computed tomography as well as by the use of intrusive analysis such as histological examination in biopsy samples. Alternatively, kidney nephrotoxicity can also be evaluated by measuring the expression of proteins whose expression is known to be associated with kidney damage, such as calbindin D-28K, kidney injury molecule-1, ostepontin, epidermal growth factor (EGF), clusterin, alpha-2 microglobulin related protein, complement component 4, vascular endothelial growth factor (VEGF), Kidney-specific Organic Anion Transporter-K1 (OAT-K1) aldolase A, aldolase B and podocin.

In another embodiment, the organ wherein toxicity is to be evaluated is heart. Cardiotoxicity can be evaluated by detecting mild blood pressure changes, thrombosis, electrocardiographic (ECG) changes, arrhythmias, myocarditis, pericarditis, myocardial infarction (MI), cardiomyopathy, cardiac failure (left ventricular dysfunction or failure) and congestive heart failure (CHF). Assays to detect cardiotoxicity are usually based on measuring potassium current blockade using heterologous expression systems, disaggregated cells, isolated tissues and the isolated intact (Langendorf-perfused) heart. In all models the effect is assessed by measurement of either ionic currents using two-electrode voltage clamp recordings or patch-clamp recordings of membrane potentials using microelectrodes or confocal microscopy.

In another embodiment, the organ wherein toxicity is to be evaluated is intestine. Intestine toxicity can be evaluated by measuring levels and/or activities of markers of intestine damage such as alkaline phosphatase, DNA content, glutathione-associated enzymes, intestinal permeability, □-glutamyl transpeptidase (GGT), quinone reductase (QR), sucrase and Ca⁺²Mg⁺²-ATPase.

In another embodiment, the organ where toxicity is evaluated is brain or the central nervous system. Markers suitable to detect damage to the brain or central nervous system includes neuron-specific enolase, S100-A, S100-B, glial fibrillary acid and myelin basic protein which can be detected either in serum or in cerebrospinal fluid.

In another embodiment, the organ where toxicity is evaluated is bone marrow. Markers suitable to detect damage to the bone marrow include serum thymidine kinase or plasma Flt-3 ligand.

In addition to be suitable for identifying cytotoxic events in a plurality of tissues, the animal models of the invention are also suitable for studying physiological events of the xenorgans. It will be appreciated that the type of physiological event that can be studied using the animals of the invention will depend on the type of tissue or organ that has been regenerated in the recipient animal. Therefore, if the xenoorgan is a liver, then the process that can be evaluated is a phase I or phase II biotransformation process and if the organ is a kidney, the interaction to be evaluated is excretion. If the organ is intestine, the interaction that is evaluated is absorption.

The invention will be illustrated by the examples shown below.

### EXAMPLES

### Example 1

### Isolation of Tissues from Human Fetal Tissues and Grafting into Immunodeficient Animals (SCID-hu Mice).

C.B-17 scid/scid mice are bred, treated with antibiotics, as is well known in the art, and used at an age 6 to 8 weeks. Anaesthesia is used during all operative procedures. The human fetal tissues are derived from curettage operation involving physical extraction without administration of prostaglandins or related drugs. The tissues are individually placed in sterile 50 ml tubes containing RPMI 1640 medium supplemented with 10% fetal bovine serum (FBS), 50 U/mL penicillin, and 50 pg/mL streptomycin at 4° C. The samples are then shipped on wet ice, received within 16 to 20 hours, and transplanted into SCID mice within 36 hours. Cells from fetal thymus or liver are tested for the presence of HIV by the DNA polymerase chain reaction as described in all cases before use. Human fetal femurs and tibias are obtained at 17 to 22 gestational weeks (gw), when intramedullary hematopoiesis is active. For construction of SCID mice implanted with HFL tissue (SCID-hu-L), HFLs of 18-22 gestational weeks were cut into fragments of −2×2×2 mm and surgically implanted into the mouse fourth mammary fat pads and in some cases also under the left kidney capsule.

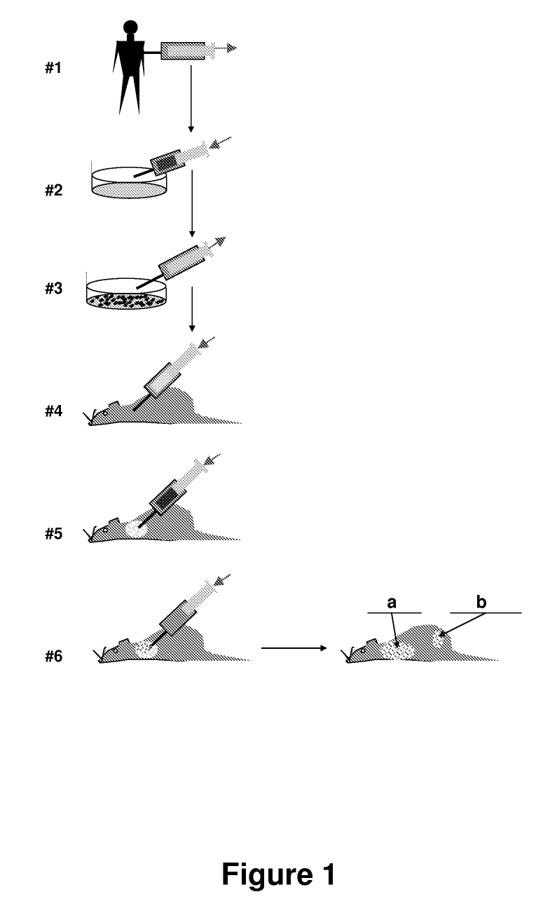

Construction of SCID mice implanted with human fetal intestinal tissues was done in a manner identical to that of SCIDhu-L. We have not observed any signs of inflammation or granulation in the lung or intestine grafts or in the surrounding murine tissues (FIG. 1 B-D). For engraftment of SCID mice with HFBM (SCID-hu-BM), human fetal femurs and tibias at 18-22 gestational weeks are put into four fragments, which are implanted individually at one or two subcutaneous sites into SCID mice. In some experiments, backbones from newborn SCID mice are implanted into mammary fat pads of SCID mice. They are cut into fragments (ca. 5×5×10 mm) and implanted subcutaneously in SCID mice. The bone grafts are removed for analysis at various intervals (×47). For irradiation, whole-body were exposed to single doses of 2 or 3 Gy using a Gamma Cell 40 irradiator from a ¹³⁷Cs source at a dose rate of 1.1 Gy/min with 30% attenuation.

### Example 2

### In Vivo Metastasis Assays in SCID-hu Mice

SCLC cells grown in vitro as suspension cultures were harvested by centrifugation, resuspended in Hanks' balanced saline solution (HBSS), assessed for cell number and viability, and injected into SCID-hu mice via lateral tail vein (experimental metastasis assay). For spontaneous metastasis assay cells were injected directly into one of the HFL grafts through a small incision in the skin.

Histology. Fragments of human grafts, murine internal organs (lungs, liver, spleen, adrenals, and sometimes additional organs), backbones, and sternums were dissected and fixed in buffered 20% (vol/vol) formalin. Bone tissues were treated with decalcifying solution (Baxter Scientific Products, McGaw Park, Ill.). After paraffin embedding, 4 □m sections are cut and stained with hematoxylin/eosin.

### Example 3

### Engraftment of MSC Cells into a Recipient Animal

There is a rational to this technique: the expected multiorgan tropism of human mesenchymal stem cells in a variety of epithelia. Seeding of human MSC on different mouse organs can be confirmed using a variety of techniques well known in the art (immunostaining, human or mouse specific PCRs, etc.). In a variation of the strochimeric technique, around the time of the MSC injection (preferentially, before), mice can be treated with physical (gamma radiation) or chemical agents with cytostatic activity, in order to suppress the mobilization and proliferation of autologous BM MSC. This treatment could enrich the engrafted human MSC population in different organs. The treatment, in addition to down-regulate the normal proliferative response of autologous BM MSC to injury, can induce a generalized injury to dividing epithelial tissues. The latter effect is likely to serve as a stimulus to homing for injected human MSC

### Example 4

### Ischemia-Reperfusion Injury of the Liver

Mice were anesthetized by i.p. injection of 100 mg/kg ketamine and 10 mg/kg xylazine and then injected s.c. with 50 □g/kg glycopyrrolate to prevent excess salivation and possible suffocation. Eye lubricant was applied to prevent ocular dehydration. Mice were placed under a heating lamp and on a 37° heating pad.

In some animals, rectal temperature was monitored with a TCAT-1A temperature control unit (Physitemp) and was found to be 35°±1° C. and did not differ between drug treatment groups. Careful monitoring of body temperature is particularly important in these experiments because A2AR agonists are vasodilators that can produce hypothermia, although this requires higher doses than were used here. We used a partial hepatic ischemia model that spares the right lobe. This model avoids intestinal congestion, sepsis, and peritonitis and is not lethal during liver ischemia times up to 60 min. Each mouse was placed in a supine position. A midline laparotomy and incision of the Linea Alba exposed the peritoneal cavity. The stomach and duodenum were displaced caudally to expose the hepatic triad and caudate lobes. The caudate lobe was separated gently from the left lobe and displaced from the right upper and lower lobes caudally to clearly view the hepatic triad above the bifurcation of right lobes, median lobe, and left lobe. A microaneurysm clip was applied to the hepatic triad above the bifurcation to clamp the flow of the hepatic artery, portal vein, and bile duct. The peritoneum was closed after superfusion with 200 □l of warm saline supplemented with 50 U/kg heparin. After 60 min of ischemia, the peritoneum was reopened, and the microaneurysm clip was removed. Immediately before reperfusion was initiated, each mouse received either a single bolus i.p. injection of 4-{3-[6-amino-9-(5-cyclopropyl-carbamoyl-3,4-dihydroxy-tetrahydrofuran-2-yl)-9H-purin-2-yl]-prop-2-ynyl}-piperidine-1carboxylic acid methyl ester (ATL313, 3 □g/kg) or an i.p. loading dose of ATL146e (1 □g/kg) and a s.c. osmotic minipump (model 1003D; ALZET) to release 10 ng/kg/min ATL146e or vehicle during the entire 24-h reperfusion period. The surgical wound was closed with metal staples, and mice were maintained on the heating pad until the anaesthetic wore off.

To assess spontaneous liver regeneration, mice are sacrificed at different times after the ischemia-reperfusion manoeuvre (24, 48, 72 hours and 1 week). Blood and tissue samples are then analyzed for signs of analytical liver functional recovery and for histological liver regeneration, respectively.

### Serum Alanine Aminotransferase (ALT) Activity

Whole blood was harvested from an incision through an axillary artery and placed on ice before centrifugation at 14,000 g for 20 min. Serum was collected and stored at −80°. ALT was determined by using an ALT kit using a plate reader. A 200-□l aliquot of a prewarmed (37°) mixture of L-alanine and □-ketoglutaric acid was added to 20 □l of undiluted and/or saline-diluted serum in a 96-well plate. After a 1-min incubation at 37, the plate was scanned at 340 nm at 9-s intervals for 60 s, and the rate of change in absorbance converted into Sigma-Frankel units (1 IU −0.482 Sigma-Frankel U).

### Example 5

### Ischemia-Reperfusion Injury of the Lung

Each animal was anesthetized with 50 mg/kg of intraperitoneal ketamine hydrochloride. A 14-gauge angiocatheter was inserted into the trachea by cervical tracheotomy. Animals were ventilated with 95% O2/5% CO2 gas at a tidal volume of 3 ml and a rate of 50 breaths/min with 3 cm of positive end-expiratory pressure using a volume-limited ventilator. After heparin (1000 U/kg) was injected, a blood sample was collected from the right common iliac artery to assess the oxygenation capacity in vivo before harvesting. A median sternotomy and thymectomy were then performed to expose the heart-lung block. A vessel cannula was placed into the main pulmonary artery through the right ventricular outflow tract and secured with 3-0 braided silk sutures through the transverse sinus. The left atrium and ventricle were amputated to vent blood. The lung was flushed with 20 ml of low potassium dextran solution at 4° C. through the main pulmonary artery from a height of 25 cm. After completion of the flush, the heart-lung block was harvested with the lungs inflated and stored for 18 h at 4° C. Other male Sprague-Dawley rats served as fresh blood donors, and heparinised blood (1000 U/kg) was collected. Each heart-lung block was mounted in a perfusion chamber maintained at 37°. The perfusion circuit was primed with 16 ml of heparinised blood adjusted to a hematocrit of 20% with modified Krebs-Henseleit buffer (NaCl: 118 mM, KCl: 4.7 mM, KH2PO4: 1.2 mM, NaHCO3: 24 mM, MgSO4 7H2O: 1.2 mM, glucose: 11.0 mM, CaCl2 H2O: 1.7 mM) containing sodium bicarbonate to maintain the pH at 7.4 to 7.5. Blood from the left atrium and ventricle was drained into the chamber and circulated to the heart-lung block through a membrane oxygenator) using a roller pump. The lung is ventilated with 95% O2/5% CO2 gas at a tidal volume of 3 ml and a rate of 50 breaths/min with 3 cm of positive end-expiratory pressure. The rate of reperfusion blood flow was remained constantly 8 ml/min during the reperfusion. Deoxygenation of the reperfusion blood in the pulmonary artery was carried out using 95% N2/5% CO2 gas delivered through the membrane oxygenator to adjust the PO2 to 40 to 50 mmHg.

### Example 6

### Ischemia-Reperfusion Injury of the Brain

Anaesthesia was induced with 4% and maintained with 3% isoflurane in air with use of a Vapomatic apparatus (Bickford Inc). Focal I/R was achieved by occluding the middle cerebral artery (MCAO) with the previously described intraluminal filament method (6-0 nylon). In brief, a midline neck incision was made, and the left external carotid artery (ECA) and left pterygopalatine artery were isolated and ligated. The internal carotid artery (ICA) was clipped at the peripheral site of the bifurcation of the ICA and the pterygopalatine artery with a small vascular clip. The blunted tip of a 6-0 nylon monofilament (0.2- to 0.22-mm-diameter tip) was advanced through the ICA to the carotid bifurcation of the ICA and ECA. The nylon thread and ECA were ligated with 6-0 silk sutures, and the ECA was cut and rotated with the nylon thread. The nylon thread was advanced until light resistance was felt, so that the distances from the nylon thread tip to the ICA-pterygopalatine artery bifurcation and the ICA-ECA bifurcation were slightly _{—}6 mm and slightly _{—}9 mm, respectively. The nylon thread was removed after 60 minute occlusion. In the sham group, these arteries were visualized but not disturbed.

### Example 7

### Ischemia-Reperfusion Injury of the Kidney

Six-week-old female SCID mice (National Cancer Institute, Frederick, Md.) are given 11 Gy-irradiation 2 h before surgery. A secondary branch of the left renal artery is separated from the vein and clamped for 15 min followed by clamp release to allow reperfusion. A group of mice also undergo right nephrectomy for evaluation of blood urea nitrogen after left renal I/R injury.

### Example 8

### Ischemia-Reperfusion Injury of Bone (Preferentially Femur).

Fifty eight male Wistar rats weighing 220-250 g were used. The animals were anesthetized with intra-peritoneal pentobarbital sodium (30 mg/Kg). Small, complementary doses of anaesthetic drug were given throughout the procedure if necessary. After anaesthesia, skin, subcutaneous tissue and muscles around the right hip joint were sectioned and only the femoral vessels and femur were left intact. One external jugular vein was dissected free and cannulated for drug injection. In the animals of the groups in which ischemia was induced, a microvascular occluding clamp was placed at the femoral artery. After 4 hours the vascular clamp was removed and blood was allowed to recirculate. Saline or AT were injected immediately before clamp removal and 99 mTc-Pyrophosfate was injected 1 hour later in treatment groups. Reperfusion was allowed for 2 hours. After this time biopsies for transition electronic microscopy were taken from soleus muscles from both hind limbs, through a posterior incision in the leg and the animals were killed with an overdose of pentobarbital. Clamping of secondary branches of arteries irrigating any given organ can be performed on a one clamped artery per organ per mouse basis or on a one clamped artery per organ several organs per mouse basis.

### Example 9

### Ischemia-Reperfusion Injury of Liver and Lung

One secondary branch of the left mouse lung and, simultaneously, one secondary branch of the main liver artery can be clamped. This doubly ischemic mouse can be used to graft, for instance, human mesenchymal stem cells to obtain a doubly ischimeric mouse, that can be subsequently transplanted orthotopically, for instance, with human colon cancer cells. This mouse would be used as a model to study the metastatic potential of human colon cancer cells to human lung and/or human liver. Hence a different method of injection (i.e. directly into the left atrium or specially left ventricle) will be preferred whenever an even multiorgan distribution of MSC is desired. In that case, techniques for assessing the contribution of different cell origins in an organ are well known in the art.

### Example 10

### Cytotoxic Injury of the Liver

As an example among many other types of tissue specific or non-specific chemical tissue injury, one can inject 0.5 ml/kg of CCl₄ into the peritoneum of 6-weeks-old Nude mice females or C57BL/6 females twice a week for 4 weeks. Liver cirrhosis results from the continuous injections of CCl₄. One day after the eighth injection, approximately 1×10⁵ GFP-positive Mesenchymal Stem Cells (MSC) are injected slowly into the tail vein using a 31G needle and a Hamilton syringe. GFP-positive MSC can be obtained from GFP transgenic mice using the purification techniques described above. After transplantation, CCl₄ injections are continued at the same dose twice a week. As a control, 1×10⁵ GFP-positive BMCs are injected into female C57BL/6 mice that had not been treated with CCl₄ (Terai S, et al. J. Biochem. (Tokyo) 2003; 134:551-558.).

### Example 11

### Isolation of Adult Human Stem Cells from Lipoaspirates.

At least 300 ml of lipoaspirate are collected into a sterile container to isolate uncultured stem cells in significant numbers (million-range). Using the technique described below, one can isolate up to 10⁷ adipose stromal stem cells with greater than 95% purity from 300 ml of lipoaspirate. However, yields can vary widely between patients.

In a first step, the lipoaspirate is extensively washed to remove the majority of the erythrocytes and leukocytes. This step is performed as follows:

1. Place a maximum of 300 ml of lipoaspirate into a used sterile medium bottle.

2. Allow the adipose tissue to settle above the blood fraction.

3. Remove the blood using a sterile 25 ml pipette.

4. Add an equivalent volume of HBSS with antibiotics and fungizone and firmly tighten the lid.

5. Shake vigorously for 5-10 seconds.

6. Place the bottle on the bench and allow the adipose tissue to float above the HBSS. This will take 1-5 min depending on the sample.

7. Carefully remove the HBSS using a 50 ml pipette.

8. Repeat the above washing procedure (steps 4 to 7) three times.

9. Medium from the final wash should be clear. If it is still red, wash again by repeating steps 4-7.

Dispersion of adipose tissue is achieved by collagenase digestion. Collagenase has the advantage over other tissue digestive enzymes that it can efficiently disperse adipose tissue while maintaining high cell viability. The collagenase digestion procedure is as follows:

1. Make up collagenase solution just prior to digestion. The final volume required is half that of the washed adipose tissue volume. Add powdered collagenase to HBSS at a final concentration of 0.2%. We dissolve the required amount of collagenase into 40 ml of HBSS, then filter sterilize into the remaining working volume. Add antibiotics and fungizone.

2. Add the washed adipose tissue to large cell culture flasks (100 ml per 162 cm2 flask).

3. Add collagenase solution.

4. Resuspend the adipose tissue by shaking the flasks vigorously for 5-10 seconds.

5. Incubate at 37° C. on a shaker for 1 to 2 h, manually shaking the flasks vigorously for 5-10 seconds every 15 min.

6. During the digestion, prepare Histopaque gradients by dispensing 15 ml of Histopaque-1077 into 50 ml tubes. Two gradients are required for each 100 ml of washed adipose tissue. The gradients must be equilibrated at room temperature before use. Prepare 200 ml of washing medium consisting of HBSS containing 2% FBS, antibiotics and fungizone.

7. On completion of the digestion period, the digested adipose tissue should have a “soup like” consistency.

8. Add FBS to a final concentration of 10% to stop collagenase activity.

After digestion, the ability of lipid-filled adipocytes to float is used to separate them from the stromal vascular fraction (SVF) as follows: The collagenase-digested tissue are dispensed into 50 ml tubes, avoiding dispensing undigested tissue, then centrifuged at room temperature at 400×g for 10 min, and, after centrifugation, the floating adipocytes, lipids and the digestion medium are aspirated with use a 50 ml pipette. The SVF pellet remaining in the tube contains erythrocytes, leukocytes, endothelial cells and stromal stem cells. Erythrocytes are removed first, using the red blood cell lysis buffer. It is essential to obtain a cell suspension free from undigested tissue and cell clumps, to effectively separate stromal stem cells from other cell types using antibody-conjugated magnetic beads. The strategies used to achieve this are separation of gross undigested tissue using gravity, straining of cells and gradient separation as is commonly performed in the art. Stromal stem cells are separated from remaining cells using magnetic cell sorting. Unwanted endothelial (CD31+) and leukocytes (CD45+) are magnetically labeled and eliminated from the cell suspension when applied to a column under a magnetic field. Magnetically labeled cells are retained in the column, while unlabeled stem cells with a CD45-CD31− phenotype pass through the column and are collected. To this end, CD31+ and CD45+ cells are labeled with FITC-conjugated anti-CD31 and anti-CD45 antibodies. The stained cells are magnetically labeled by the addition of anti-FITC-conjugated magnetic microbeads. This approach presents the advantage that cell purity after separation can be assessed by flow cytometry or fluorescence microscopy.

The success of obtaining pure stromal stem cell samples of high purity varies between donors. It is therefore important to assess the purity of the sample using a fluorescence-based assay. Fluorescence microscopy is usually sufficient to evaluate purity. A more accurate assessment can be made by flow cytometry; however, this assay requires many more cells. Under white light, stromal stem cells have an evenly round phenotype while endothelial cells have an irregular shape. Also, under epifluorescence, one should determine the percentage of fluorescent cells under 5 fields of view. The average represents the percentage of contamination of non stem cells in the sample.

Culture can also be used to further validate the successful isolation of stromal stem cells from adipose tissue using the above procedure. Stromal stem cells, when cultured, adhere to plastic and acquire a fibroblastic-like morphology. It may take several days before all adherent cells change their morphology. In our own experience, approximately 50% of cells isolated as above will plate under the correct culture conditions. However, plating efficiency can vary substantially between donors. To encourage adherence, one should plate isolated stem cells in medium containing 50% FBS in a volume sufficient to smear the medium across the surface of a cell culture flask, then incubate in a humidified incubator at 37° C., 5% CO2. It usually takes several days before those cells which form a fibroblastic morphology start dividing.

Generation of stable adipose stem cell lines is required to evaluate their differentiation capacity and proliferative ability. We have kept lines of stem cells generated by the above isolation method for longer than 6 months without loss due to senescence.

After at least 7 days of culture, the culture medium is replaced with DMEM:F12 containing antibiotics, 20% FBS and no fungizone, then the cells are sub-cultured using standard methods of trypsinization after a further week of culture to form a stable cell line. To evaluate the “stemness” of adipose stem cell lines established, it is recommended to differentiate the cells towards various mesodermal lineages. For adipogenic differentiation, cell cultures are incubated in DMEM:F12 medium containing 10% FBS, 0.5 μM 1-methyl-3 isobutylxanthine, 1 μM dexamethasone, 10 μg/ml insulin and 100 μM indomethacin for 3 weeks. Then the medium is changed every 4 days. To visualize lipid droplets, the cells are with 4% formalin and stain with Oil-Red O. For osteogenic differentiation, the cells are incubated in DMEM:F12 medium containing 10% FBS, 100 nM dexamethasone, 10 mM β-glycerophosphate and 0.05 mM L-ascorbic acid-2-phosphate for 3 weeks. Then the medium is changed every 4 days. Mineralization of the extracellular matrix is visualized by staining with Alizarin Red.

### Example 12

### Making of Chimeric Humanized SCID Mice (Strochimeric Mice):

The mice will be made using a three step process:

1) SCID mice will be submitted to different types of organ injury. A preferred injury method is of the ischemia reperfusion (IRI) type. Afferent arteries to the organ(s) of interest will be clamped for various times in order to induce ischemia.
2) Once the clamp is released, these mice will be systemically injected with human precursor cells (i.e. adult stem cells or hASC), that have previously been immortalized with an immortalizing gene+green GFP construct. These cells should preferentially set, proliferate and differentiate in the ischemic tissue in an attempt to reconstruct the damaged organ.
3) Successful humanized strochimeras will be transplanted with a variety of human cells (tumor derived cell lines, metastasis derived cell lines, cell lines derived from micrometastasic cells, etc.). These cells have been previously immortalized with a gene construct that also expresses red GFP.

Analysis in these experiments will include:

Pathology staining after IRI (step 1) to assess the level of organ damage,

Transilumination after step 2 to check for the presence of green GFP positive cells in the organ that have been submitted to IRI,

Immuno-histochemistry using human specific antibodies after step (2) to study the degree of differentiation of human adult stem cells within the mouse organ,

Transilumination (every day) to check the implantation, invasion and dissemination of human cancer cell lines (red), and finally immunohistochemistry (depending on transillumination results or when there is a macroscopic mass or in any case 4 weeks after injection in the orthotopic site and eight weeks in the metastatic site) to microscopically asses those last three features.

Controls will include:

In step (1), there will be control mice injected with human ASC but without being previously submitted to IRI. Also there will be mice injected with mock after being submitted to IRI.

In step (3), there will also be mice injected with normal human epithelial cells (ideally primary cultures) to assess specificity of tumor implants.

Variations of step 1 include pre-treatment with radiation to avoid bone marrow mobilization of autologous mesenchymal stem cells.

A different application of this technology is the analysis of the feasibility of making a humanized mouse liver to use in toxicity, metabolism, catabolism or other pharmacological studies. Here, step 1 will be essentially the same. The hepatic artery or one of its main branches will be clamped and released to inflict IRI (other methods of provoking injury, like chemical toxicity, can be used). This will be followed by systemic (or local intraarterial) injection of hASC. Experimental controls and checking analysis could be similar as in the tumor models.

1. A method for obtaining an animal model for tumor metastasis comprising at least one cellular component from at least one other animal from a different species, said method comprising the steps of:
i) inflicting damage leading to cellular death to at least one tissue or organ in a recipient animal,
ii) implanting into the recipient animal a composition comprising precursor or stem cells from a donor animal from a different species wherein said precursor or stem cells are capable of differentiating into the same tissue, tissues, organ or organs that have been damaged in the recipient animal in step (i),
iii) allowing the cells implanted in step (ii) to differentiate into the at least one tissue or at least one organs that has or have been damaged in step (i) and
iv) implanting into the recipient animal cells derived from a tumor isolated from the donor animal or cells of a tumor cell line which derive from a tumor isolated from the donor animal.

2. The method according to claim 1 wherein the recipient animal is selected from the group consisting of rodents, mice, immunodepressed animals, SCID mice, and embryonic stage animals.

3. The method according to claim 1 wherein the donor animal is a human.

4. The method according to claim 1 wherein the damage in step (i) is caused by a damaging modality selected from the group consisting of chemical means, cytotoxic compositions, physical means and ischemia/reperfusion.

5. The method according to claim 1 wherein the damage is limited to one tissue or organ.

6. The method according to claim 1 wherein the damage is applied to more than one tissue or organ.

7. The method according to claim 1 wherein the cells implanted in step (ii) are systemically administered or orthotopically administered.

8. The method according to claim 1 wherein the composition used in step (ii) is blood, a bone marrow aspirate or a purified precursor cell preparation wherein the precursor cells are selected from the group consisting of adult stem cells, immortalized stem cells, epithelial stem cells, embryonic stem cells, umbilical cord stem cells, mesenchymal stem cells, stem cells that have been genetically modified to express a reporter gene that allows tracing the stem cells or any cell originating from said stem cells, and mixtures including two or more of the foregoing.

9. The method according to claim 1 wherein the recipient animal is treated between steps (i) and (ii) to increase repopulation of stem cells to the place of lesion.

10. The method according to claim 9 wherein the treatment to increase repopulation is selected from the group of injection into the injured organ of individual molecular cues, injection into the injured organ of acellular extracts from injured tissues and a combination thereof.

11. The method according to claim 1 wherein the recipient animal is also implanted after step (i) with fibroblasts, mesenchymal stem cells or a mixture thereof from the donor animal.

12. The method according to claim 11 wherein the fibroblasts, mesenchymal stem cells or mixture thereof are orthotopically administered at the site of tissue damage or are administered systemically.

13. The method according to claim 11 wherein the mesenchymal stem cells have been immortalized prior to implantation.

14. The method according to claim 11 wherein the fibroblasts, mesenchymal stem cells or mixture thereof are administered simultaneously with the precursor cells or prior to the administration of the precursor cells.

15. The method according to claim 1 wherein the recipient animal is also implanted at the site of tissue damage with an extracellular matrix.

16. The method according to claim 15 wherein the extracellular matrix is selected from the group consisting of natural matrices that occur in the damaged tissue and artificial cellular matrices.

17. The method according claim 1 wherein the recipient animal is treated between steps (i) and (ii) to prevent mobilization of endogenous stem cells.

18. The method according to claim 17 wherein the treatment to prevent mobilization of endogenous stem cells is done by irradiation or using nicotine receptor antagonists.

19. The method according to claim 1 wherein the material implanted in step (iv) is selected from the group of:
a. circulating tumor cells
b. cell lines derived from the immortalization of circulating tumor cells
c. micrometastatic tumor cells
d. lines derived from the immortalization of micrometastatic tumor cells
e. cell lines derived from immortalized tumor cells that had been previously purified from solid tumors
f. primary tumor cells from solid tumors
g. a piece of fresh tumor that has been resected from a solid tumor
h. primary tumor cells
i. cell lines derived from immortalized cells that had been previously purified from clinical metastasis (i.e. the PC3 cell line) and
j. any combination of a) to i).

20. A method according to claim 1 wherein the cells implanted in step (iv) have been genetically modified with a reporter gene so as to express a protein that allows tracing the cells implanted in step (iv).

21. A method according to claim 1 wherein the implantation step (iv) is carried out by a method selected from the group of tail vein injection, intracardial injection, intraperitoneal injection and orthotopic implantation into an organ.

22. A method for obtaining an animal model for tumor metastasis comprising at least one cellular component from at least one other animal from a different species, said method comprising the steps of:
(i) implanting into a recipient animal a composition comprising mesenchymal stem cells from a donor animal from a different species and allowing the implanted cells to differentiate into cells of mesenchymal tissues,
(ii) implanting into the recipient animal a composition comprising precursor or stem cells from a donor animal from the same species than the donor animal used in step (i), so that the precursor or stem cells are capable differentiate into the same tissue, tissues, organ or organs that have been populated by the mesenchymal stem cells implanted in step (i) and
(iii) implanting orthotopically into at least one tissue or organ that has been populated by mesenchymal stem cells implanted in step (i) cells derived from a tumor isolated from the donor animal or cells of a tumor cell line which derive from a tumor isolated from the donor animal.

23. The method according to claim 22 wherein the mesenchymal stem cells are primary cells or immortalized cells.

24. The method according to claim 22 wherein the mesenchymal stem cells are administered systemically, or orthotopically into a tissue or organ of choice.

25. The method according to claim 22 wherein the mesenchymal stem cells implanted in step (i) are administered simultaneously with fibroblasts.

26. The method according to claim 22 wherein the material implanted in step (iii) is selected from the group of:
i) circulating tumor cells
ii) cell lines derived from the immortalization of circulating tumor cells
iii) micrometastatic tumor cells
iv) lines derived from the immortalization of micrometastatic tumor cells
v) cell lines derived from immortalized tumor cells that had been previously purified from solid tumors
vi) primary tumor cells from solid tumors
vii) a piece of fresh tumor that has been resected from a solid tumor
viii) primary tumor cells
ix) cell lines derived from immortalized cells that had been previously purified from clinical metastasis (i.e. the PC3 cell line) and
x) any combination of i) to ix).

27. A non-human animal produced by the method of claim 1.

28. A non-human animal according to claim 27 wherein the damage in step (i) has been applied to the liver, to the kidney, to the brain, to the bone or to more than one of said organs simultaneously.

29. A non-human animal according to claim 27 wherein the recipient animal is selected from the group consisting of rodents, mice, immunodepressed animals and SCID mice.

30. A non-human animal according to claim 27 wherein the donor animal is a human.

31. A method of studying metastasis in an animal subject, comprising using as said animal subject an animal according to claim 27.

32. A method for the identification of a substance capable of inhibiting and/or preventing metastasis of tumor cells, comprising the steps of:
i) administering a test substance to a non-human animal according to claim 27; and
ii) measuring inhibitory and/or preventive effect of the test substance on metastasis.

33. A method for evaluating efficiencies of treatment against metastasis of tumor cells, said method comprising the steps of:
i) applying a treatment to the non-human animal according to claim 27 and
ii) comparing the size and/or extent of metastasis, and/or symptoms resulted from metastasis, with a control animal.

34. A method for identifying genes which are involved in metastasis progression comprising the steps of
i) Preparing a non-human animal according to claim 27 wherein the tumor cells implanted in step (iv) have been transfected with a cDNA whose effect in metastasis wants to be studied, and
ii) Monitoring the appearance of metastatic lesions in the organ which has been regenerated in step (iii),
wherein the appearance of a higher number of metastatic lesions in the animal in comparison with a control animal which has received non-transformed tumor cells is indicative that the candidate gene is involved in metastasis progression.

35. A method for determining the effect of a test substance on metastasis, comprising the steps of:
i) administering a test substance to a non-human metastasis model animal according to claim 27; and
ii) comparing the size and/or extent of metastasis, and/or symptoms resulted from metastasis, with a control animal.

36. A method of evaluating the interaction or the effects of a compound of interest with or on organ or tissue that has been regenerated in a recipient animal comprising at least one cellular component from at least one other animal from a different species, said method comprising use of a recipient animal obtained by a process comprising the steps of:
i) inflicting damage leading to cellular death of at least one tissue or organ in a recipient animal;
ii) implanting into the recipient animal a composition comprising precursor or stem cells from a donor animal from a different species wherein said precursor or stem cells are capable of differentiating into the same tissue, tissues, organ or organs that have been damaged in the recipient animal in step (i); and
iii) allowing the cells implanted in step (ii) to differentiate into the tissue, tissues, organ or organs that have been damaged in step (i) so as to regenerate at least in part the organ that has been damaged in step (i), and
administering the compound of interest to said recipient animal for said evaluation.

37. A method according to claim 36 wherein the animal is selected from the group consisting of rodents, mice, immunodepressed animals, SCID mice and recipient animals in the embryonic stage.

38. A method according to claim 36 wherein the precursor or stem cells implanted in step (ii) are of human origin.

39. A method according to claim 36 wherein the damage in step (i) is caused by a damaging modality selected from the group consisting of chemical means, cytotoxic compositions, physical means and ischemia/reperfusion.

40. The method according to claim 36 wherein the damage is limited to one tissue or organ.

41. The method according to claim 36 wherein the damage is applied to more than one tissue or organ.

42. The method according to claim 36 wherein the cells implanted in step (ii) are systemically administered or orthotopically administered.

43. The method according to claim 36 wherein the composition used in step (ii) is blood, a bone marrow aspirate or a purified precursor cell preparation wherein the precursor cells are selected from the group of adult stem cells, epithelial stem cells, embryonic stem cells, umbilical cord stem cells and mesenchymal stem cells or a mixture thereof.

44. The method according to claim 36 wherein the stem cells used in step (ii) have been immortalized.

45. The method according to claim 36 wherein the stem cells used in step (ii) have been genetically modified so as to express a reporter gene that allows tracing the stem cells or any cell originating from said stem cells.

46. The method according to claim 36 wherein the recipient animal is treated between steps (i) and (ii) to increase repopulation of stem cells from distant organs to the place of lesion.

47. The method according to claim 46 wherein the treatment to increase repopulation is selected from the group of injecting into the injured organ of individual molecular cues, injection into the injured organ of acellular extracts from injured tissues and a combination thereof.

48. The method according to claim 36 wherein the recipient animal is also implanted after step (i) with fibroblasts, mesenchymal stem cells or a mixture thereof from the donor animal.

49. The method according to claim 48 wherein the fibroblasts, mesenchymal stem cells or mixture thereof are orthotopically administered at the site of tissue damage or administered systemically.

50. The method according to claim 48 wherein the mesenchymal stem cells have been immortalized prior to implantation.

51. The method according to claim 48 wherein the fibroblasts, mesenchymal stem cells or mixture thereof are administered simultaneously with the precursor cells, or prior to the administration of the precursor cells.

52. The method according to claim 36 wherein the recipient animal is also implanted at the site of tissue damage with an extracellular matrix.

53. The method according to claim 52 wherein the extracellular matrix is a natural matrix which occurs in the damaged tissue, or an artificial cellular matrix.

54. The method according to claim 36 wherein the recipient animal is treated between steps (i) and (ii) to prevent mobilization of endogenous stem cells.

55. The method according to claim 54 wherein the treatment to prevent mobilization of endogenous stem cells is done by irradiation.

56. The method according to claim 36 wherein the effect that is to be evaluated is toxicity.

57. The method according to claim 56 wherein the organ wherein toxicity is evaluated is selected from the group of brain, liver, heart, lung, intestine, kidney and bone marrow.

58. The method according to claim 36 wherein the organ that has been regenerated in the recipient animal is liver and the interaction that is evaluated is a phase I or phase II biotransformation process.

59. The method according to claim 36 wherein the organ that has been regenerated in the recipient animal is kidney and the interaction that is evaluated is excretion.

60. The method according to claim 36 wherein the organ that has been regenerated in the recipient animal is intestine and the interaction that is evaluated is absorption.

61. The method according to claim 36 wherein the organ that has been regenerated in the recipient animal is bone marrow and the interaction that is evaluated is absorption.
